(19) United States
(12) Patent Application Publication
(10) Pub. No.: US 2009/0326529 A1
(43) Pub. Date: Dec. 31, 2009

(54) METHOD AND APPARATUS FOR REDUCING IMAGE ARTIFACTS IN ELECTRONIC ABLATION IMAGES

(76) Inventors: Christopher L. Brace, Madison, WI (US); Fred T. Lee, JR., Madision, WI (US)

Correspondence Address: WISCONSIN ALUMNI RESEARCH FOUNDATION, C/O BOYLE FREDRICKSON S.C, 840 North Plankinton Avenue, Milwaukee, WI, 53203, US

(21) Appl. No.: 12/488,895

(22) Filed: Jun. 22, 2009

Related U.S. Application Data
(60) Provisional application No. 61/074,367, filed on Jun. 20, 2008.

(51) Int. Cl. A61B 18/14

(52) U.S. Cl. 606/41

(57) ABSTRACT

At least one electrode lead outside the body and leading between an RF ablation power source and the unshielded probes in the patient is shielded to substantially eliminate artifacts during concurrent electronic imaging and RF ablation.

### CROSS REFERENCE TO RELATED APPLICATION

This application claims the benefit of U.S. provisional application Ser. No. 61/074,367 filed Jun. 20, 2006 hereby incorporated by reference.

### STATEMENT REGARDING FEDERALLY SPONSORED RESEARCH OR DEVELOPMENT

This invention was made with United States government support awarded by the following agencies:
NIH CA108869

The United States government has certain rights to this invention.

### BACKGROUND OF THE INVENTION

The present invention relates to medical procedures involving an exposed radio frequency electrode, such as RF ablation, and, in particular, to a method of providing electronic medical imaging during such radio frequency procedures.

Electronic medical imaging, for example, digital radiography, computed tomography (CT), ultrasound imaging, and magnetic resonance imaging (MRI), employ sophisticated electronic sensors and computational systems to produce superior images of in vivo tissue.

In an example computed tomography (CT) system, as known in the art, typically include an x-ray source collimated to form a beam (either a cone beam or a fan beam) extending along an axis through an imaged object to be received by an x-ray detector array. The x-ray source and detector array are oriented to lie within the x-y plane of a Cartesian coordinate system, termed the “imaging plane”.

The x-ray source and detector array may be rotated together on a gantry within the imaging plane, around the imaged object, and hence around the z-axis of the Cartesian coordinate system. Rotation of the gantry changes the angle at which the fan beam intersects the imaged object, termed the “gantry” angle.

The detector array is comprised of detector elements each of which measures the intensity of transmitted radiation along a ray path extending from the x-ray source to that particular detector element. At each gantry angle, a projection is acquired comprised of intensity signals from each of the detector elements. The gantry is then rotated to a new gantry angle and the process is repeated to collect a number of projections along a number of gantry angles to form a tomographic projection set.

The tomographic projection set may be reconstructed mathematically, for example using the “filtered back projection” algorithm, to yield a cross-sectional image of the imaged object viewable perpendicular to the direction of the x-ray beams. The ability to reconstruct a cross-sectional image from the edge-wise projections relies on a mathematically balanced augmentation and cancellation among the different projections of the projection set. Imaging conditions that upset this balance create severe image artifacts in the forms of streaks and stars that obscure clinical information. Generally, the cause of the artifacts is not easily deduced from observation of the artifacts themselves.

Common sources of artifacts include: (1) under-sampling of the x-ray signal, (2) failure to obtain projections over sufficient angular range, (3) axial or irregular movement of the patient, x-ray tube, or x-ray detector, (4) partial volumes imaged at only some angles, (5) “beam-hardening” caused by different attenuation of high and low frequency x-rays, and (6) radio opaque structures in the region of interest that create strong “shadows”.

Radio frequency thermal ablation uses metallic electrodes inserted into tissue, for example a tumor, to produce electrical heating of the tissue to destroy the tumor. Desirably, such ablation may be performed during imaging of the tissue in order to monitor the size of the ablated region. Computed tomography or other electronic medical imaging techniques would be well suited to such monitoring of ablation; however, current experience using CT during radio frequency thermal ablation, for example, is that the ablation process produces severe and obscuring streak artifacts. Similar artifacts have been discovered in digital radiography and Doppler ultrasound images.

One study of these artifacts, described in: Brennan, “CT Artifact Introduced by a Radio Frequency Ablation”, AJR 2006; 186:S284-S286 (2006), noted that the artifacts are linked to the application of power during ablation and speculated that electromagnetic cross talk was interfering with CT data acquisition in some undetermined way. This paper suggests that the artifacts may be the result of the omission of beam-hardening corrections in fast CT fluoroscopy making the CT system more susceptible to interference or thinner detectors being more susceptible to interference. Discouragingly, however, this study found that these severe artifacts, precluding useful examination of the ablation zone and procedural monitoring, were not appreciably changed with changes in ablation current but could be decreased only by stopping the ablation process or by increasing x-ray tube current substantially.

### SUMMARY OF THE INVENTION

The present inventors have determined that the artifacts produced during radio frequency ablation or other similar procedures, such as, cardiac ablation, electrocautery, varicose vein ablation, are electromagnetic interference transmitted from unshielded ablation leads, but more importantly, that even though the ablation electrode must be exposed to tissue and thus unshielded, and further even though substantial current is conducted through the unshielded patient during the ablation process, shielding of only the portion of the cables leading to the electrode outside of the body appears to be sufficient to substantially eliminate artifact generation even for substantial ablation currents.

Specifically then, the present invention provides a method of monitoring radio frequency ablation comprising placing at least one electrode into conductive contact with tissue of a patient in a region to be ablated and applying a radio frequency electrical signal to the electrode to ablate the tissue in the region, the electrode receiving the radio frequency electrical signal from a remote radio frequency generator via a first conductor connecting between the electrode and the remote radio frequency generator. An electrical return from the tissue to the remote radio frequency generator is provided via a second conductor providing a return path from the patient to the remote radio frequency generator wherein the first and second conductors are shielded by placement within or integrated into a conductive electrical shield for substantially the entire length of the conductor between the patient and the remote radio frequency generator. Concurrently with the application of the radio frequency electrical signal, an electronic medical image of the region being ablated is acquired.

It is thus one feature of at least one embodiment of the invention to exploit the recognition of the disproportionate influence of electrical interference from the leads to permit artifact free imaging during the ablation process.

The electrical shield may be a first and second conductive tube separating the first and second conductors respectively, each tube providing a high-frequency path to ground.

It is thus one feature of at least one embodiment of the invention to provide a system that permits greatest freedom in placement of the electrode and the return conductors.

The tubes may be conductive braids.

It is thus one feature of at least one embodiment of the invention to provide leads that are flexible and thus amenable to use with the patient in an imaging device.

The first and second conductors are center conductors of standard coaxial cables and the first and second conductive tubes are coaxial shields surrounding the center conductors separated by an electrical dielectric providing a characteristic impedance of the coaxial cable.

It is thus one feature of at least one embodiment of the invention to permit the use of standard cabling that is commercially available.

Alternatively, the electrical shield may provide a conductive tube surrounding the first conductor and wherein the second conductor is a portion of the conductive tube.

It is thus one feature of at least one embodiment of the invention to provide a configuration with fewer leads reducing the possibility of entanglement and a decreasing their radiative length.

The electrical shield comprises a conductive tube surrounding the first and second conductors. The first and second conductors may be twisted about each other.

It is thus one feature of at least one embodiment of the invention to promote interference cancellation by exploiting the countervailing current flows in the first and second conductors.

These particular features and advantages may apply to only some embodiments falling within the claims and thus do not define the scope of the invention.

### BRIEF DESCRIPTION OF THE DRAWINGS

FIG. 1 is a representation of a CT system, in phantom, showing the internal x-ray tube and detector, and a radio frequency ablation system positioned near the CT system for concurrent imaging and ablation;

FIG. 2 is a cross-sectional view through a patient in the CT system showing insertion of an ablation electrode for ablation of a tumor and the relative location of the detector during a portion of the imaging process;

FIG. 3 is a schematic representation of a first shielding technique;

FIG. 4 is a schematic representation of a second, balanced, shielding technique;

FIG. 5 is a schematic representation of a third reduced conductor shielding technique;

FIGS. 6a and 6b are pictorial representations of x-ray CT images showing the streak artifacts generated without the present invention and their substantial reduction when the present invention is employed;

FIGS. 7a and 7b are pictorial representations of digital x-ray images showing the streak artifacts generated without the present invention and their substantial reduction when the present invention is employed.

### DETAILED DESCRIPTION OF THE INVENTION

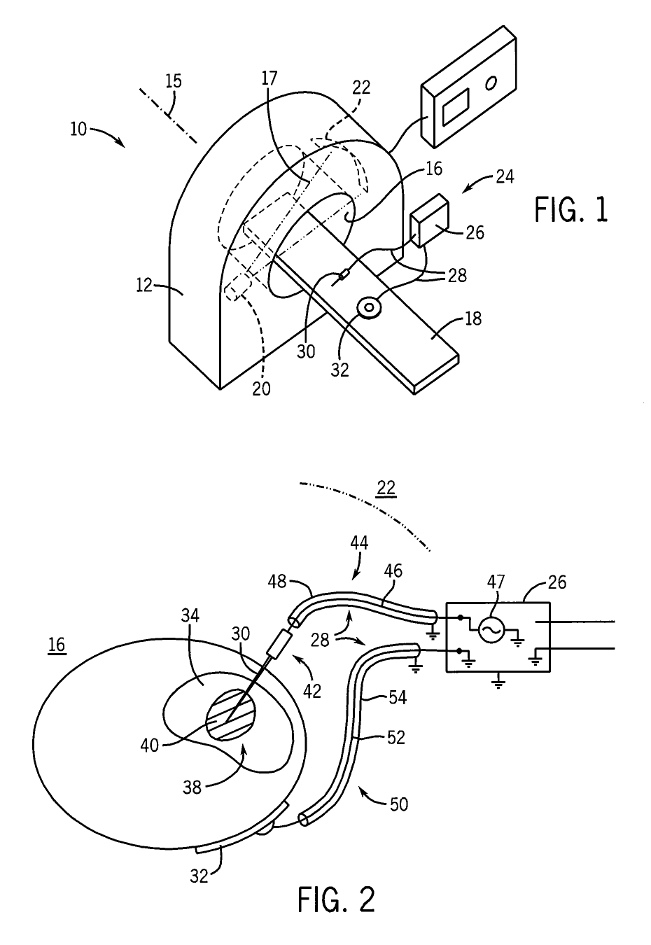

Referring now to FIG. 1, a CT machine 10 includes a gantry 12 having a bore 16 extending along an axis 15 to receive a patient (not shown) supported on a patient table 18 extending along the axis 15. Inside the gantry 12, an x-ray tube 20 may project an x-ray beam 17 to an x-ray detector 22 opposed across the bore 16. The x-ray tube 20 and detector 22 may orbit about the bore 16 (on a rotation axis aligned with axis 15) to obtain projections through the patient on the patient table 18 at a variety of angles in a plane perpendicular to axis 15.

A radio frequency ablation system 24 may be positioned near the bore 16 to provide a source of radio frequency power through a generator 26 connected to leads 28. In a “monopolar” mode, one lead 28 is connected to a radio frequency probe 30 and the other to a conductive ground pad 32. Generally, the invention is equally applicable to a bipolar system where current flows between two probes 30 and 30′ or portions of a single probe having two mutually insulated portions (not shown in FIG. 1).

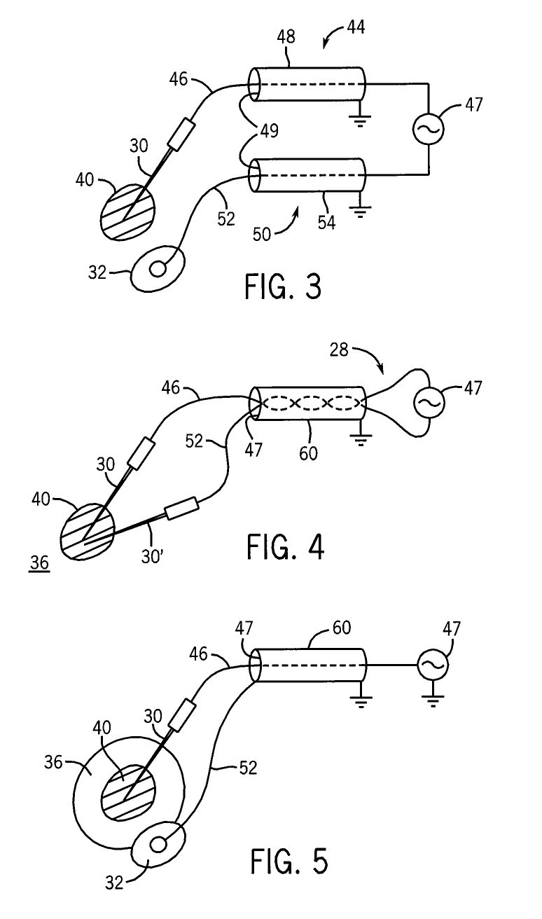

Referring now to FIGS. 2 and 3, the probe 30 may be inserted into an organ 34 within a patient 36 positioned within the bore 16 for imaging. The probe 30 may have a small cross-section and a pointed tip to be inserted by piercing the skin so that a proximal end 38 of the probe 30 may embed in or near a tumor at an ablation region 40. The outer surface of the probe 30 in the ablation region 40 is electrically conductive to form an ohmic contact with the region 40 for the introduction of electrical current into the region 40 and heating thereof.

A distal end 42 of the probe 30 extends out of the patient 36 to be connected to a center conductor 46 of a shielded cable 44 providing outer shield 48 coaxially around the center conductor 46. The center conductor 46 of the shielded cable 44 provides electrical communication between the probe 30 and the generator 26. At the generator 26, the center conductor 46 is connected to a radio frequency power source 47, for example, providing 500 kHz radio frequency power and, in any case, radio frequency electrical power less than 300 MHz.

One end of the shield 48 (conveniently at the generator 26) is connected to ground (or any low impedance voltage reference) to create a constant potential shield around conductor 46 reducing the radiation of electromagnetic energy. The shield 48 may be connected to a metallic housing of the generator 26 providing an enclosed Faraday shield for the generator 26 which may also be grounded or connected to any low impedance voltage reference. Grounding for this purpose refers to a low impedance connection at the frequency of the generator 26 that need not be ohmic.

Similarly, a ground pad 32, providing a broad area of contact to the patient 36, may be attached to a center conductor 52 of a separate shielded cable 50 providing a coaxial outer shield 54. As is understood in the art, the ground pad 32 provides a broad area of contact to the skin of the patient 36 allowing electrical flow between the probe 30 and the ground pad 32 without significant heating near the ground pad 32 during high heating and ablation in the region 40.

The center conductor 52 of the shielded cable 50 provides electrical communication between the ground pad 32 and the generator 26, at which the center conductor 52 joins to the generator ground and the shield 54 is connected to a ground or another point of low impedance constant voltage.

This configuration may be used in the monopolar mode, described above, with a probe 30 and ground pad 32, or (as shown) used in a bipolar mode with a first probe 30 and similar second probe 30′ both placed within the patient 36 with current flowing between them to create the ablation region 40 as described below.

Referring now to FIG. 4, in an alternative balanced shielding configuration, the conductors 46 and 52 may be placed in close proximity (for example, loosely twisted) so that their countervailing currents tend to cancel. They are then together placed in surrounding coaxial shield 60 attached to a source of constant potential with respect to the patient 36 as described above with respect to shields 54 and 48. This configuration may be used in the monopolar mode, described above, with a probe 30 and ground pad 32, or (as shown) used in a bipolar mode with a first probe 30 and similar second probe 30′ both placed within the patient 36 with current flowing between them to create the ablation region 40. The probes 30 and 30′ may be needle probes as described above or umbrella probes or other types of percutaneous electrodes.

Referring now to FIG. 5, in an alternative reduced lead shielding configuration, the conductor 46 is placed in coaxial shield 60 attached to a source of constant potential with respect to the patient 36 as described above with respect to shields 54 and 48. In this case, the conductor 52 may be integrated with the shield 60 to reduce the need for a separate conductor 52 along the shielded length of the conductor 46. This configuration may be used in the monopolar mode, described above, with a probe 30 and ground pad 32, or (as shown) used in a bipolar mode with a first probe 30 and similar second probe 30′ both placed within the patient 36 with current flowing between them to create the ablation region 40.

The probes 30 and 30′ may be needle probes as described above or umbrella probes or other types of percutaneous electrodes.

The conductors 46 and 52 may be separated from the shields 48, 54 or 60 by means of an intervening electrical insulator 49. In the embodiments of FIGS. 3 and 5, the shield 60 and the conductors 46 may be realized by a standard coaxial cable such as provides a radio frequency transmission line RG type cables or maybe a standard shielded cable without transmission line properties. The shields 48, 54 and 60 may be braided conductive wire to provide flexibility to the cables 44.

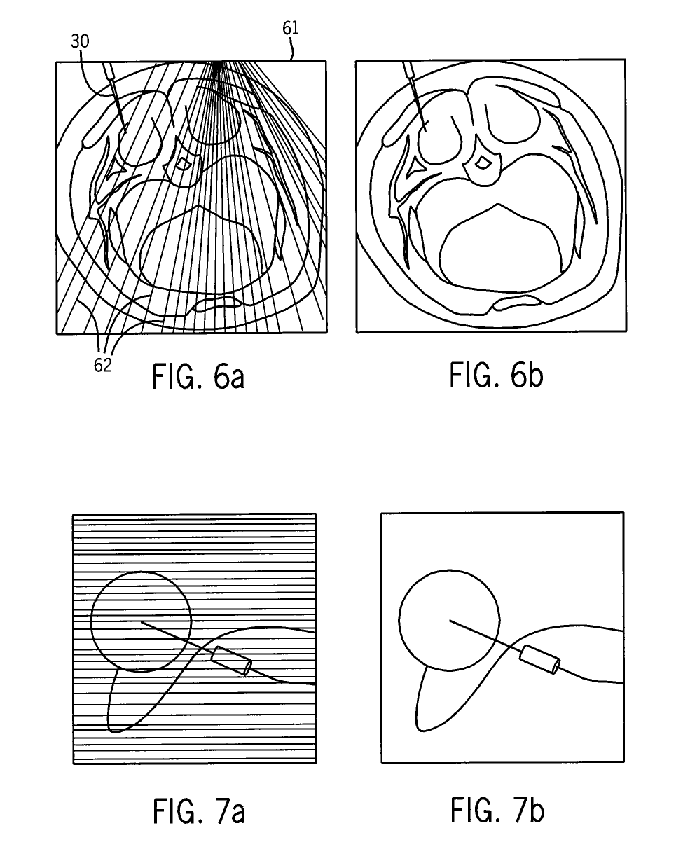

Referring now to FIG. 6a, an example CT image 61 shows streak artifacts 62 emanating from an arbitrary point removed from the ablation of probe 30 reminiscent of beam-hardening artifacts caused by a spectral change in x-ray energy as it passes through a patient. Shielding of the conductors 46 and 52 in the embodiment of FIG. 3 produces the image of FIG. 6b in which the artifacts are substantially eliminated.

Referring now to FIG. 7a, an example digital x-ray image shows horizontal banding. Shielding of the conductors 46 and 52 in the embodiment of FIG. 3 produces the image of FIG. 6b in which the banding is substantially eliminated.

While the present invention has been described in the context of computed tomography and digital radiography, it will be understood that the same technique may be applicable to other imaging modalities including not only computed tomography, ultrasound, magnetic resonance imaging, and the like, but also, for example, electrically sensitive computer monitors attached to devices such as endoscopes and the like. The fact that limited shielding of cables leading to an unshielded patient can provide pronounced attenuation of electrical interference may also be useful for non-imaging applications such as the acquisition of ECG signals etc. The shielding techniques described could include non-tubular shielding arrangements such as tightly twisted pair were shielding is provided by close proximity of counteracting currents. Further, it will be understood that other devices applying electrical energy to the body when concurrent imaging must be conducted using electronic imaging devices, may benefit from the present invention. Such devices may include, for example, those providing for cardiac ablation, electrocautery, varicose vein ablation, etc.

It should be understood that the invention is not limited in its application to the details of construction and arrangements of the components set forth herein. The invention is capable of other embodiments and of being practiced or carried out in various ways. Variations and modifications of the foregoing are within the scope of the present invention. It also being understood that the invention disclosed and defined herein extends to all alternative combinations of two or more of the individual features mentioned or evident from the text and/or drawings. All of these different combinations constitute various alternative aspects of the present invention. The embodiments described herein explain the best modes known for practicing the invention and will enable others skilled in the art to utilize the invention.

We claim:

1. A method of monitoring radio frequency ablation comprising:
(a) placing at least one electrode into conductive contact with tissue of a patient in a region to be ablated;
(b) applying a radio frequency electrical signal to the electrode to ablate the tissue in the region, the electrode receiving the radio frequency electrical signal from a remote radio frequency generator via a first conductor connecting between the electrode and the remote radio frequency generator;
(c) providing an electrical return from the tissue to the remote radio frequency generator via a second conductor providing a return path from the patient to the remote radio frequency generator; wherein the first and second conductors are shielded by placement within or integrated into a conductive electrical shield for substantially the entire length of the conductor between the patient and the remote radio frequency generator; and
(c) concurrently with step (b), acquiring an electronic medical image of the region being ablated.

2. The method of claim 1 wherein an electrical shield comprises a first and second conductive tube separating the first and second conductors, respectively, each tube providing a high-frequency path to ground.

3. The method of claim 2 wherein the tubes are conductive braids.

4. The method of claim 2 wherein the first and second conductors are center conductors of standard coaxial cables and the first and second conductive tubes are coaxial shields surrounding the center conductors separated by an electrical dielectric providing a characteristic impedance of the coaxial cable.

5. The method of claim 1 wherein the electrical shield comprises a conductive tube surrounding the first conductor and wherein the second conductor is a portion of the conductive tube.

6. The method of claim 5 wherein the first and second conductive tubes are metallic braids.

7. The method of claim 1 wherein the electrical shield comprises a conductive tube surrounding the first and second conductors.

8. The method of claim 7 wherein the first and second conductors are twisted about each other.

9. The method of claim 1 wherein the radio frequency generator produces a signal having a frequency in the radio frequency domain of less than 300 MHz

10. The method of claim 1 wherein the radio frequency generator produces a signal having a frequency substantially equal to 500 kHz.

11. The method of claim 1 wherein the return path is provided in part by a ground pad attached to the skin of the patient providing a large contact area with the patient allowing ablation at the region without burning of the skin at the contact area, the ground pad communicating via the second conductor with the remote radio frequency generator.

12. The method of claim 1 wherein the return path is provided in part by a second electrode inserted into tissue of the patient near the region to be ablated.

13. The method of claim 1 wherein the placing of the electrode pierces the skin to reach an internal tumor site.

14. The method of claim 1 wherein the electrode is unshielded.
